(19) United States
(12) Patent Application Publication
(10) Pub. No.: US 2026/0294410 A1
(43) Pub. Date: Oct. 1, 2026

(54) LOCKING CONNECTION SYSTEMS AND METHODS

(71) Applicant: ECA Medical Instruments, Thousand Oaks, CA (US)

(72) Inventors: David SKINLO, Thousand Oaks, CA (US); Dan Anvi-Baifus, Thousand Oaks, CA (US); Zachary Hixson, Thousand Oaks, CA (US)

(21) Appl. No.: 19/631,869

(22) Filed: Mar. 27, 2026

Related U.S. Application Data
(60) Provisional application No. 63/779,071, filed on Mar. 27, 2025.

(51) Int. Cl. A61B 17/00

(52) U.S. Cl. CPC A61B 17/00; A61B 2017/00464

(57) ABSTRACT

A system for connecting a first component to a second component is disclosed. In certain embodiments, the first component comprises a handle and the second component comprises a medical instrument. The system includes a handle defining a receiving pocket and a locking feature, and an instrument having a metal working end and a proximal mating component configured for insertion into the receiving pocket. The proximal mating component of the instrument includes a non-circular geometry that restricts rotational movement and a body portion configured to engage the receiving pocket to limit lateral movement. The locking feature engages the proximal mating component to restrict axial movement and secure the instrument within the handle. In some embodiments, the handle is formed of a polymer material and the instrument is formed of metal. A plurality of instruments having standardized mating components may be interchangeably used with the handle.

### CROSS-REFERENCE TO RELATED APPLICATION

This application claims the priority benefit of U.S. Provisional Pat. App. No. 63/779,071 to Skinlo, et al., filed Mar. 27, 2025 and entitled “Locking Connection for Medical Instruments,” which is fully incorporated by reference herein in its entirety.

### BACKGROUND OF THE DISCLOSURE

### Field of the Disclosure

The present disclosure relates generally to modular connection systems and methods for releasably or permanently coupling one component to another. More specifically, the disclosure relates to connection interfaces for coupling a working component, attachment, or tool to a support component, holder, handle, or base structure.

### Description of the Related Art

In many industries, it is desirable to selectively couple interchangeable components using a secure connection that resists movement in multiple degrees of freedom. Such systems may be useful in medical, dental, laboratory, industrial, consumer, cosmetic, cleaning, food-service, precision assembly, or maintenance applications. Conventional assemblies often require dedicated unitary devices or fail to provide a standardized interface that permits secure interchangeability or modularity.

For example, medical and surgical procedures frequently require the use of specialized instruments designed for cutting, grasping, retracting, or otherwise manipulating tissue. Traditionally, such instruments are manufactured as unitary devices in which the functional metal working end is permanently affixed to a dedicated handle. These instruments are commonly fabricated from stainless steel or other durable metals to withstand repeated sterilization and use. While effective, this construction results in relatively high manufacturing costs due to the material requirements, machining processes, and assembly involved.

In recent years, there has been increased interest in reducing costs and improving efficiency in medical settings, particularly with respect to single-use or limited-use instruments. However, many existing instruments remain expensive to produce and procure, especially when each variation of a tool requires a fully assembled, standalone device. This can lead to increased inventory burdens, as healthcare providers must stock multiple complete instruments that differ only in the configuration of their functional, tool end.

Contemporary medical tool systems have not achieved widespread standardization. The lack of a common interface for coupling instrument components limits flexibility and prevents cost savings that could otherwise be realized through interchangeable parts.

Accordingly, there exists a need for an improved system that allows a variety of metal instrument working ends to be selectively and securely attached to a standardized handle. In particular, a system utilizing a standardized plastic handle could reduce material costs, simplify manufacturing, and enable disposable or semi-disposable use, while still maintaining the strength and precision required of the metal working components. A standardized attachment mechanism would further promote interoperability, reduce inventory complexity, and provide a more economical solution for healthcare providers.

### SUMMARY OF THE DISCLOSURE

Disclosed herein are systems, methods, and devices for connecting a first component to a second component, such as connecting a working tool to a handle.

One embodiment of a medical instrument system according to the present disclosure includes a handle, which includes a body defining a pocket and a first mating component within the pocket; and an instrument, which includes a tool end and a second mating component configured to mate with the first mating component and including non-circular geometry.

One embodiment of a modular medical instrument system according to the present disclosure includes: a plurality of instruments, each having a tool end and a first mating component opposite the tool end and a plurality of handles each having a body defining a pocket and a second mating component within the pocket configured to mate with the first mating component and restrict at least one of lateral, rotational, and axial movement of the instrument relative to the handle.

One embodiment of a method of assembling a medical instrument system according to the present disclosure includes: providing a handle comprising a pocket and a first mating component, providing an instrument comprising a tool end and a second mating component configured to mate with the first mating component, and inserting the instrument in the pocket such that the first mating component and the second mating component mate and prevent at least one of lateral, axial, and rotational movement of the instrument relative to the handle.

This has outlined, rather broadly, the features and technical advantages of the present disclosure so that the detailed description that follows may be better understood. Additional features and advantages of the disclosure will be described below. It should be appreciated by those skilled in the art that this disclosure may be readily utilized as a basis for modifying or designing other structures for carrying out the same purposes of the present disclosure. It should also be realized by those skilled in the art that such equivalent constructions do not depart from the teachings of the disclosure as set forth in the appended claims. The novel features, which are believed to be characteristic of the disclosure, both as to its organization and method of operation, together with further features and advantages, will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE FIGURES

FIG. 1 shows an exploded top, front, right-side perspective view an embodiment of a medical instrument and handle system incorporating features of the present disclosure;

FIG. 2 shows an exploded, cross-sectional view of the medical instrument and handle system of FIG. 1; and

FIG. 3 shows top, front, right-side perspective view of the medical instrument and handle system of FIG. 1.

FIG. 4 shows a top, left-side perspective view of another embodiment of a medical instrument according to the present disclosure.

FIG. 5 shows a top, right-side perspective view of the medical instrument of FIG. 4.

FIG. 6 shows a top, right-side perspective view of another medical instrument and handle system according to the present disclosure, which incorporates the medical instrument of FIGS. 4 and 5.

### DESCRIPTION OF THE DISCLOSURE

In the following detailed description, numerous specific details are set forth in order to provide a more thorough understanding of embodiments incorporating features of the present disclosure. However, it will be apparent to one skilled in the art that devices and methods according to the present disclosure can be practiced without necessarily being limited to these specifically recited details.

Throughout this disclosure, embodiments and examples illustrated should be considered as exemplars, rather than as limitations on the present disclosure. As used herein, the term “invention,” “device,” “method,” “present invention,” “present device,” or “present method” refers to any one of the embodiments of the disclosure described herein, and any equivalents. Furthermore, reference to various feature(s) of the “invention,” “device,” “method,” “present invention,” or “present device,” or “present method” throughout this document does not mean that all claimed embodiments or methods must include the referenced feature(s).

Furthermore, any element in a claim that does not explicitly state “means for” performing a specified function, or “step for” performing a specific function, is not to be interpreted as a “means” or “step” clause as specified in 35 U.S.C. § 112, for example, in 35 U.S.C. § 112(f) or pre-AIA 35 U.S.C. § 112, sixth paragraph. In particular, the use of “step of” or “act of” in the claims herein is not intended to invoke the provisions of 35 U.S.C. § 112.

It is also understood that when an element or feature is referred to as being “on” or “adjacent” to another element or feature, it can be directly on or adjacent the other element or feature or intervening elements or features may also be present. It is also understood that when an element is referred to as being “attached,” “connected” or “coupled” to another element, it can be directly attached, connected or coupled to the other element or intervening elements may be present. In contrast, when an element is referred to as being “directly attached,” “directly connected” or “directly coupled” to another element, there are no intervening elements present.

Furthermore, relative terms such as “left,” “right,” “front,” “back,” “top,” “bottom'” “forward,” “reverse,” “clockwise,” “counter-clockwise,” “outer,” “inner,” “above,” “upper,” “lower,” “below,” “horizontal,” “vertical,” and similar terms, have been used for convenience purposes only and are not intended to imply any particular fixed direction. Instead, they are used to describe a relationship of one element to another. Terms such as “higher,” “lower,” “wider,” “narrower,” and similar terms, may be used herein to describe angular relationships. It is understood that these terms are intended to encompass different orientations of the elements or system in addition to the orientation depicted in the figures.

Although ordinal terms, e.g., first, second, third, etc., may be used herein to describe various elements or components, these elements or components should not be limited by these terms. These terms are only used to distinguish one element or component from another element or component. Thus, a first element or component discussed below could be termed a second element or component without departing from the teachings of the present disclosure.

The terminology used herein is for describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises,” “comprising,” when used herein, specify the presence of stated features, integers, steps, operations, elements, and/or components, but do not preclude the presence or addition of one or more other features, integers, steps, operations, elements, components, and/or groups thereof.

Embodiments as described in the present disclosure can be described herein with reference to view illustrations that are schematic in nature. As such, the actual thickness of elements can be different, and variations from the shapes of the illustrations as a result, for example, of manufacturing techniques and/or tolerances are expected. Thus, the elements illustrated in the figures are schematic in nature and their shapes are not intended to illustrate the precise shape of a region and are not intended to limit the scope of the disclosure. Further, it is understood that, while embodiments of the present disclosure comprise various shapes, these shapes are not exhaustive, and other shapes are possible.

It is understood that when a first element is referred to as being “between” or “interposed between” two or more other elements, the first element can be directly between the two or more other elements or intervening elements may also be present between the two or more other elements. For example, if a first element is “between” or “interposed between” a second and third element, the first element can be directly between the second and third elements with no intervening elements, or the first element can be adjacent to one or more additional elements with the first element and these additional elements all between the second and third elements.

In some embodiments, the systems described herein may be understood as including a first component and a second component configured to be attached, connected, or coupled to one another through cooperating mating structures. The terms “first” and “second” are used for convenience only to distinguish one component from another and are not intended to imply any particular order or limitation. In the illustrated embodiments, the first component is shown as a handle and the second component is shown as an instrument or tool. However, the disclosed pocket, mating component, locking feature, and motion-restricting interface structures may be employed in other corresponding component pairs.

Assembly of a second component into a first component, such as assembly of a medical instrument (e.g., made of metals or plastics) into “J” or “D” connection handles (e.g., made of metals or plastics, like polymers) can be a unilateral assembly that ensures a secure connection between the components resulting in an assembly that behaves homogenously.

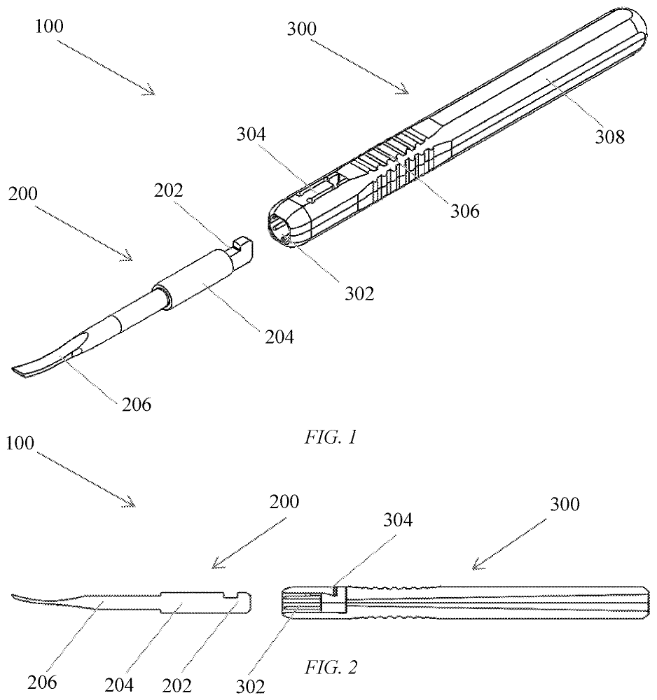

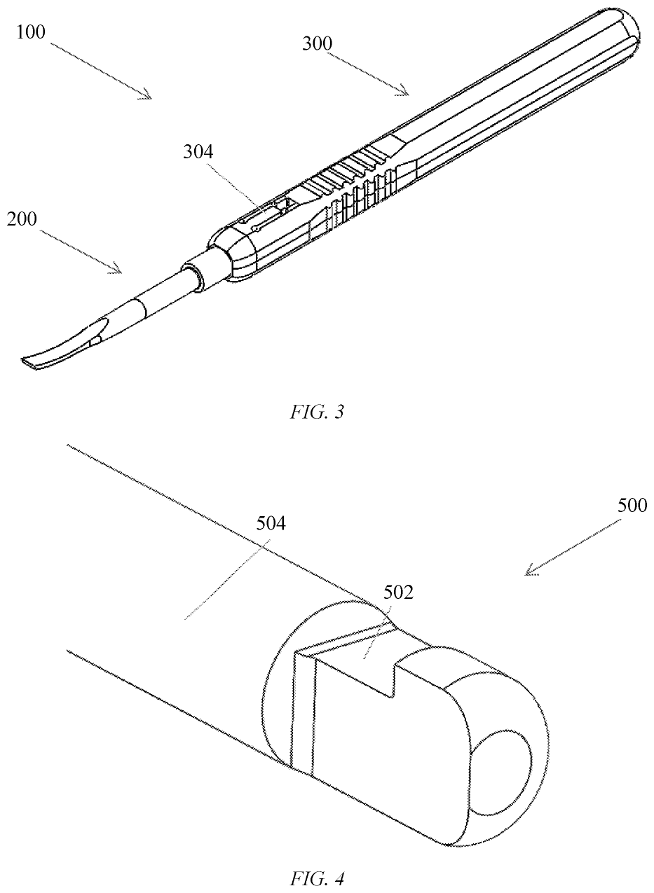

FIGS. 1-3 show one embodiment of a medical instrument system 100 according to the present disclosure. The medical instrument system 100 can, in some embodiments, include an instrument 200 and a handle 300. In this embodiment, the handle 300 may be referred to as a first component and the instrument 200 may be referred to a second component. In some embodiments, the proximal end of the instrument 200 is inserted into a pocket 302 on the distal end of the handle 300. Mating components 202,304 are designed to ensure that the instrument 200 cannot move relative to the handle 300 and cannot be removed once inserted completely. In some embodiments, the instrument 200 can be removed once inserted, but to do so requires substantial force such that the instrument 200 and the handle 300 will not be separated during use, but may be separated when not in use. In a specific embodiment, the mating component 304 is designed to be broken when not in use to release the instrument 200 from the handle 300.

As used herein “pocket” may refer to any receiving portion, receptacle, opening, recess, slot, channel, bore, socket, cavity, passage, or other structure configured to receive at least a portion of a mating component or body portion of a corresponding component. Such a pocket or receiving portion may be open or partially enclosed, may extend fully or partially through a body, and may have any suitable depth, profile, or cross-sectional shape. Such a pocket or receiving portion may also guide, align, support, and/or position the corresponding component and may cooperate with one or more interface features to constrain relative movement between components.

In some embodiments according to the present disclosure, the instrument 200 can contain a mating cylindrical body 204 located distally from the mating component 202, as well as a tool end 206.

Restricting motion of the components 200,300 relative to each other is achieved by the three design aspects which, taken together, provide multi-axis constraint, resulting in a stable, unitary assembly. First, restricting rotation of the components relative to each other is achieved by the rectangular cross section of the mating component 202, shown as a “J” shaped proximal portion of the instrument 200, within a corresponding rectangular slot in the handle 300, shown in FIG. 2. Second, restriction of toggle or bending relative to each other is achieved by a tight fit between the mating cylindrical body 204 and the pocket 302. Third, linear relative motion is restricted by a locking clip 304 on the plastic handles that mates with the mating component 202.

In some embodiments according to the present disclosure, the handle 300 can include a textured portion 306 and a smooth portion 308. The textured portion 306 and the smooth portion 308 allow for comfortable and confident manipulation of the tool end 206 vis-à-vis the handle 300.

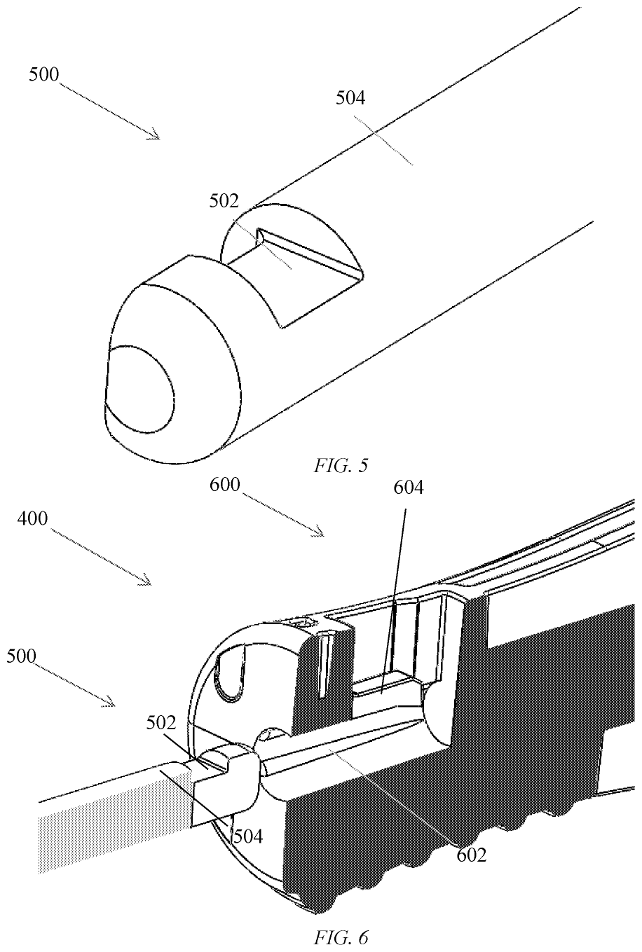

FIGS. 4-6 show another embodiment of a medical instrument system 400 according to the present disclosure. Like the medical instrument system 100, the medical instrument system 400 can, in some embodiments, include an instrument 500 and a handle 600. In this embodiment, the handle 600 may be referred to as a first component and the instrument 500 may be referred to as a second component. The instrument 500 can contain a mating component 502, a cylindrical body 504, and a tool end 506 (not pictured), which may be the same or similar to the tool end 206. The handle 600 can contain a pocket 602 and a locking clip 604.

Restricting motion of the components 500,600 relative to each other is achieved by the three design aspects which, taken together, provide multi-axis constraint, resulting in a stable, unitary assembly. First, restricting rotation of the components relative to each other is achieved by the rectangular cross section of the mating component 502, shown as a “D” shaped proximal portion of the instrument 500, within a corresponding rectangular slot in the handle 600, shown in FIG. 6. Second, restriction of toggle or bending relative to each other is achieved by a tight fit between the mating cylindrical body 504 and the pocket 602. Third, linear relative motion is restricted by a locking clip 604 on the plastic handles that mates with the mating component 502.

In some embodiments according to the present disclosure, the handle 300,600 can be made of plastic, polymeric materials, composites, metals, and other materials known in the art, or combinations thereof, and the instrument 200,500 can be made of metal, composite materials, ceramics, or other materials suitable for medical use. In some embodiments of systems according to the present disclosure, many instruments 200,500 may be manufactured with different tool ends 206 but substantially identical mating components 202,502. Many handles 300,600 can be manufactured in a standardized format with mating components 304,604. Such systems simplify manufacturing and reduce costs associated with medical instruments. Additionally, in some embodiments, the handles 300,600 can be disposable or semi-disposable to reduce or eliminate sterilization burdens.

It is generally understood that one or both mating components may comprise one or more interface features configured to engage one another and to constrain relative movement between corresponding components, including at least rotational movement. While “D” and “J” shaped geometries are used herein as examples, it is understood that other interface features and noncircular geometries are possible, such as for example, polygonal, keyed, splined, or otherwise irregular cross-sectional configurations, such geometries being configured to inhibit rotational movement between the instrument and the handle, or more generally between the second component and the first component. In some embodiments, the mating components may comprise complementary interface features configured to engage one another, including protrusions, recesses, keyed surfaces, splines, flats, grooves, or combinations thereof. The mating components may be formed integrally with the instrument 200,500 and handle 300,600, respectively, or more generally with the second component and the first component, or may comprise separate components coupled thereto.

In some embodiments, the interface between the instrument 200,500 and the handle 300,600 is configured to restrict movement in multiple degrees of freedom, including rotational, lateral, and axial movement. Such restriction may be achieved through any combination of geometric interlocking, surface engagement, frictional forces, or mechanical locking features, and need not be limited to the specific structures illustrated herein.

Similarly, while a locking clip 304,604 is disclosed as an example, other mechanisms are possible resist axial separation of the instrument 200,500 from the handle 300,600, including but not limited to clips, detents, interference fits, snap-fit features, latches, spring-biased elements, threaded engagements, bayonet connections, frictional interfaces, or combinations thereof. The locking feature may be integral with the handle 300,600, the instrument 200,500, or provided by cooperative engagement between features of the handle 300,600 and the instrument 200,500.

Likewise, although a cylindrical body is disclosed as an example, lateral or transverse stability between the instrument and the handle may be achieved through engagement of one or more surface regions, which may include cylindrical, tapered, polygonal, or irregular geometries, and may include line contact, surface contact, or distributed contact regions.

The connection systems and methods disclosed herein may be used with medical instruments, dental instruments, grooming tools, cosmetic applicators, cleaning implements, laboratory tools, inspection devices, hand tools, precision tools, consumer products, kitchen implements, or other devices in which a functional member is coupled to a support member, handle, holder, or receiver.

The working portion may comprise a cutting feature, gripping feature, scraping feature, probing feature, applicator feature, cleaning feature, polishing feature, mixing feature, dispensing feature, sensing feature, or other functional structure.

It is understood that embodiments herein may be used in disposable, reusable, or semi-disposable systems.

It is understood that embodiments presented herein are meant to be exemplary. Although the present disclosure has been described in detail with reference to certain preferred configurations thereof both in the specification and in the claims, other versions are possible. Embodiments of the present disclosure can comprise any combination of compatible devices/features described herein and/or shown in the figures, and these embodiments should not be limited to those expressly illustrated and discussed. For instance and not by way of limitation, the appended claims could be modified to be multiple dependent claims so as to combine any combinable combination of elements within a claim set, or from differing claim sets. Claims depending on one independent claim could be modified so as to depend from a different independent claim. Therefore, the spirit and scope of the disclosure should not be limited to the versions described above.

While the foregoing written description of the disclosure enables one of ordinary skill to make and use what is considered presently to be the best mode thereof, those of ordinary skill will understand and appreciate the existence of variations, combinations, and equivalents of the specific embodiments, methods, systems, and examples herein. The disclosure should therefore not be limited by the above-described embodiments, methods, systems, and examples. Furthermore, certain terminology has been used for the purposes of descriptive clarity, and not to limit the present disclosure. It is therefore intended that the following appended claims include all such alterations, modifications and permutations as fall within the true spirit and scope of the present disclosure. No portion of the disclosure is intended, expressly or implicitly, to be dedicated to the public domain if not set forth in the claims.

We claim:

1. A medical instrument system comprising:
a handle comprising:
a body defining a pocket; and
a first mating component within said pocket;
an instrument comprising:
a tool end; and
a second mating component configured to mate with said first mating component and comprising non-circular geometry that restricts at least one of axial, rotational, and lateral movement of said instrument relative to said body.

2. The medical instrument system of claim 1, wherein said non-circular geometry comprises a J-shaped configuration.

3. The medical instrument system of claim 1, wherein said non-circular geometry comprises a D-shaped configuration.

4. The medical instrument system of claim 1, wherein said body further comprises a cylindrical portion.

5. The medical instrument system of claim 1, wherein said first mating component comprises a locking clip.

6. The medical instrument system of claim 5, wherein said locking clip is configured to permit removal of said instrument from said handle when said medical instrument system is not in use.

7. The medical instrument system of claim 5, wherein said locking clip comprises a frangible portion configured to break to permit removal of said instrument.

8. The medical instrument system of claim 5, wherein said locking clip is configured to permanently secure said instrument to said handle.

9. The medical instrument system of claim 1, wherein said handle comprises a textured portion.

10. A modular medical instrument system comprising:
a plurality of instruments, each comprising a tool end and a first mating component opposite said tool end;
a plurality of handles comprising:
a body defining a pocket; and
a second mating component within said pocket configured to mate with said first mating component and restrict at least one of lateral, rotational, and axial movement of said instrument relative to said handle.

11. The system of claim 10, wherein at least two of said plurality of instruments comprise different tool ends.

12. The system of claim 10, wherein said first mating component comprises non-circular geometry.

13. The system of claim 10, wherein said second mating component comprises a locking clip.

14. The system of claim 10, wherein said handle comprises a polymer material.

15. The system of claim 10, wherein said instrument comprises metal.

16. A connection system comprising:
a first component comprising a pocket and a first mating component; and
a second component comprising a functional portion and a second mating component configured to engage said first mating component,
wherein engagement of said first mating component and said second mating component constrains relative movement between said first component and said second component in at least a rotational direction, and
wherein said connection system further comprises an axial retention feature on at least one of said first component and said second component, said axial retention feature configured to resist withdrawal of said second component from said first component.

17. The system of claim 16, wherein at least one of said first component and said second component comprise non-circular cross-sectional geometry configured to constrain said rotational movement.

18. The system of claim 17, wherein said non-circular cross-sectional geometry comprises a D shape.

19. The system of claim 17, wherein said non-circular cross-sectional geometry comprises a J shape.

20. The connection system of claim 16, wherein said axial retention feature comprises a locking clip.
